(19) United States
(12) Patent Application Publication
(10) Pub. No.: US 2006/0253193 A1
(43) Pub. Date: Nov. 9, 2006

(54) Mechanical means for controlling blood pressure

(76) Inventors: Samuel Victor Lichtenstein, Vancouver (CA); Daniel Gelbart, Vancouver (CA)

Correspondence Address: DANIEL GELBART, 4706 DRUMMOND DR., VANCOUVER, BC, V6T-1B4, CA

(21) Appl. No.: 11/119,766

(22) Filed: May 3, 2005

(51) Int. Cl. A61M 1/10

(52) U.S. Cl. 623/3.1; 600/16

(57) ABSTRACT

Making the volume of the arterial system increase elastically with blood pressure reduces high systolic blood pressure peaks and can also assist the pumping function of the heart by reducing load on the heart. This volumetric elasticity is achieved by the action of a spring controlling a volume. The spring can be implanted on the outside of the aorta and the volume is elastically controlled by squeezing the aorta or the spring can be installed inside an elongated elastic sealed vessel inserted into the aorta. Such a sealed vessel will respond to increase in blood pressure by reducing its volume, thus preventing the blood pressure from rising. When the latter method is used the device can be inserted into the aorta via a major artery without the need for major surgery. In both cases the device is powered by the blood pressure itself and requires no other energy source or control circuits.

### FIELD OF THE INVENTION

The invention relates to controlling high blood pressure by a simple implantable device and its potential for decreasing cardiac afterload and increasing coronary perfusion.

### BACKGROUND OF THE INVENTION

High blood pressure is a very common disorder primarily caused by the major arteries losing flexibility over the years or smaller arterioles increasing vascular resistance. As the heart pumps the blood into the aorta, the aorta and other arteries behave as an elastic vessel expanding in order to absorb the newly injected volume of blood before it spreads in the body. This volumetric elasticity prevents the pressure from rising too high. With age and other factors arterioles increase their resistance and the large arteries loose their ability to expand in response to the pressure increase, resulting in high systolic pressure The ability to elastically increase the volume in response, to a pressure increase is referred to as “volumetric elasticity” in this disclosure.

Traditionally high blood pressure is treated by medication, with the well-known disadvantages of having to take regular medication, side effects, costs, need for continuous supply etc. For patients equipped with devices generally known as “heart-assist” devices or artificial hearts, these devices conceivably can be programmed to regulate the blood pressure and indeed such active means of controlling blood pressure and flow are well known in the literature. In this disclosure the term “active device” refers to a device that is powered by a power source, either internal or external to the body. Such devices usually also have electronic controls for regulating and monitoring their operation; some are fully programmable. As these devices require significant power, supplied by batteries or alternating current source, they have the major disadvantage of needing to keep these batteries charged either by surgical replacement or external means or limit the mobility of the patient. Another disadvantage is that all these devices require major surgery to be installed in the body.

A passive device (i.e. having no power source) is highly preferred because of simplicity, reliability, cost, and eliminating the need for a power source, which is generally the weak link in active devices.

Passive devices have been used to assist the heart but mainly in the form of components such as heart valves or external braces to strengthen and support the heart against the internal pressures. In general the purpose of the external wraps and braces applied to a weakened heart is to reduce the “volumetric elasticity” of the heart, as if the heart volume expands too easily with pressure it can not reduce it volume to expel the blood against the back pressure of the aorta.

One object of the invention is to bring back the required “volumetric elasticity” to the arteries, in order to limit pressure peaks and emulate a blood circulation system of a healthy person. Another object is to limit and regulate the blood pressure by using a flexure based passive device. Flexure based devices have no sliding parts that can wear out. Still another object is to make such a passive device small and simple, so it can be implanted in the body (either inside or outside the aorta) by minimally invasive surgery. Still another objective is a device, which can be implanted either inside or outside the aorta or other large arteries but does not require puncturing the wall of the artery. Further objects and advantages of the invention will become apparent by reading the disclosure in conjunction with the drawings.

### SUMMARY OF THE INVENTION

Making the volume of the arterial system increase elastically with blood pressure reduces high systolic blood pressure peaks. This volumetric elasticity is achieved by the action of a spring controlling a volume. The spring can be implanted on the outside of the aorta and the volume is elastically controlled by squeezing the aorta or the spring can be installed inside an elongated elastic sealed vessel inserted into the aorta. Such a sealed vessel will respond to increase in blood pressure by reducing its volume, thus preventing the blood pressure from rising. When the latter method is used the device can be inserted into the aorta via a major artery without the need for major surgery. In both cases the device is powered by the blood pressure itself and requires no other energy source or control circuits.

### BRIEF DESCRIPTION OF THE DRAWINGS

FIG. 1 shows a view of an external elastic device installed on the aorta.

FIG. 2-a shows a cross section of the external elastic device of FIG. 1 at the moment of highest systolic pressure.

FIG. 2-b shows a cross section of the external elastic device of FIG. 1 at the moment of the lowest pressure.

FIG. 3 shows a view of an internal elastic device installed inside the aorta, the wall of the aorta is shown cut open for clarity.

FIG. 4-a shows a cross section of the internal elastic device of FIG. 3 at the moment of highest systolic pressure.

FIG. 4-b shows a cross section of the internal elastic device of FIG. 3 at the moment of lowest pressure.

FIG. 5 shows an alternate embodiment of the internal elastic device, the wall cut open to reveal the internal spring.

FIG. 6 shows a different alternate embodiment of the internal elastic device, the wall cut open to reveal the internal spring.

### DETAILED DESCRIPTION OF THE INVENTION

The inventions restores the lost volumetric elasticity to the arteries by first decreasing the volume inside the arteries, allowing it to increase elastically as the pressure goes up. Since the amount of blood pumped out by the heart into the aorta during each contraction is about 60 cubic centimeter (cc), even a change in volume as small as 5 cc during each heartbeat will have an effect on systolic blood pressure and increasing the aortic volume by 10 cc will reduce an abnormally high blood pressure to a normal value. Since the change in volume of the blood in the aorta is comparable to the volume pumped out with each contraction, changing the volume of the aorta by 10 to 20% is sufficient to prevent high blood pressure and can be accomplished by an external or internal elastic device. An external device is simpler and can achieve a higher volumetric elasticity, however it requires opening the chest cavity to implant. An internal elastic device can be inserted into the aorta through a major artery such as in the leg, similar to balloon insertion done today for angioplasty, but the device is more complicated. In some circumstances the patient is undergoing surgery in the area of the aorta for other reasons, therefore an external elastic device can be installed at the same time and may be preferred to an internal device. In both devices the materials used and design details are critical for two main reasons:
1. The device flexes each time the heart beats thus the lifetime should be in the order of a few billion flexing cycles without a failure.
2. Materials should be compatible with blood and body tissue and in the case of an internal device capable of true hermetic seal because of the vacuum used (details are given later).

The fatigue life of an elastic element made of metal can be made practically infinite if proper design is used. For example, the hairspring (the escapement spring) in a mechanical watch beats about five times faster than the human heart and lasts a lifetime. This is possible because of phenomena known as “endurance limit” in highly elastic metals such as heat-treated steels. This means that if a spring is stressed below a certain stress level (about 50% of the ultimate tensile strength for hardened steel) fatigue life will be billions of cycles and failures will be random. In order to further reduce chances of random fatigue failure in the preferred embodiment the stress levels in the material are kept below 30% of the ultimate tensile stress and the stressed areas are free of scratches, as effects and scratches can start a fatigue failure. The design theory for the elastic elements required for this invention is well known to mechanical engineers and is also available online (including software for design optimization), for example at: <http://www.engrasp.com/products/etbx/library/fm/index.jsp>Regarding the second requirement of compatibility with the human body as well as hermetic seal, the best materials are spring tempered (hard) stainless steels, both series 300 and series 400 and plated heat-treated beryllium copper. There are many other materials compatible with the human body but most have a lower endurance limit. This subject is also well known in the medical art as many implants are used today. The preferred embodiment for an external solution:

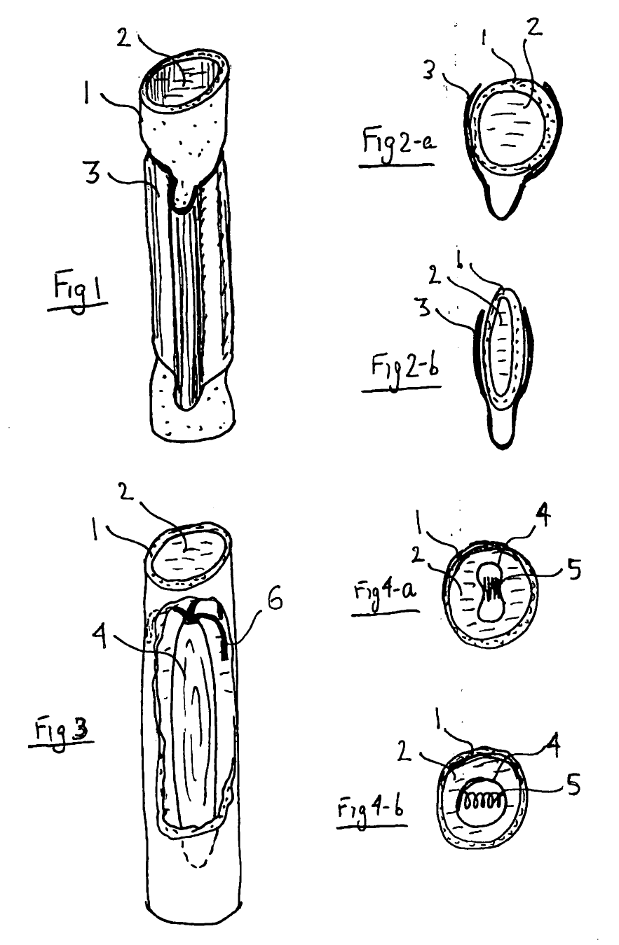

Referring to FIG. 1, the aorta 1 is filled with blood 2. A flexible clip 3 is clamped on the outside of the aorta 1. The systolic blood pressure forces the aorta 1 into circular shape (to maximizes volume) as shown in FIG. 2a. During diastolic pressure, spring clip 3 returns to its natural shape shown in FIG. 2b. The blood volume in the aorta in this state is significantly less than in the systolic state shown in FIG. 2a. This change of volume emulates the elasticity of a healthy aorta and reduces systolic pressure. Clip 3 can be made of metal or a polymeric material. In the preferred embodiment it is made of spring temper stainless steel (300 series or 400 series) or nickel-plated heat-treated beryllium copper. Typical thickness is 0.1-0.3 mm and edges should be rounded and beaded to avoid sharp edges inside the body. The metal can be treated in order to promote bonding to body tissue. Clip 3 can also be coated with a polymeric elastomer such as silicone rubber to avoid sharp edges. The spring constant should be chosen that increasing the systolic pressure from 100 to 200 mmHg should spread the spring open by about 1 cm. This created an aortic volume change of about 25 cc for a spring about 20 cm long. The preferred embodiment for an internal solution:

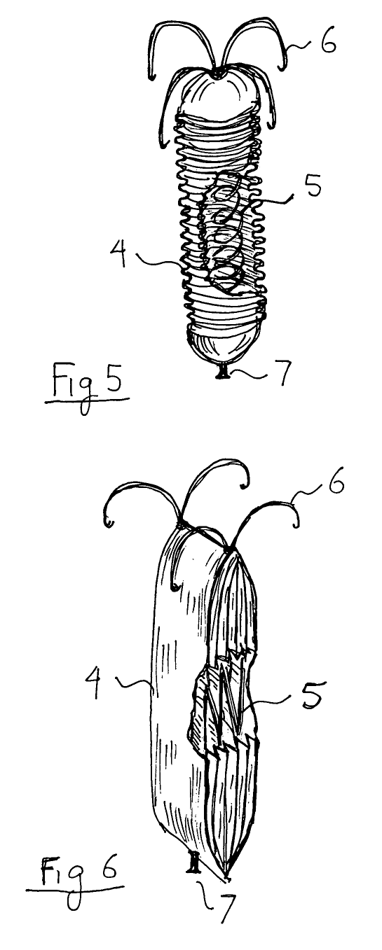

The embodiment, suitable for insertion via the artery (i.e. not requiring major surgery) is shown in FIG. 3. A hollow flexible sealed vessel 4 is inserted into the aorta. As blood pressure increases, vessel 4 is flattened as it has less volume when its cross section is elongated as shown in FIG. 4a than when it returns to its natural circular cross section as shown in FIG. 4b. As blood pressure goes from diastolic to systolic vessel 4 changes its shape from the shape shown in FIG. 4b to FIG. 4a. The change in shape significantly decreases the volume of vessel 4, emulating the elasticity of a healthy aorta and reduces systolic pressure. Vessel 4 can be made of metal or a polymeric material. In the preferred embodiment it is made of spring temper stainless steel (300 series or 400 series) or nickel-plated beryllium copper (heat treated for maximum elasticity). The metal surface can be treated in order to avoid thrombus formation. The interior of vessel 4 has to be placed under full or partial vacuum, as systolic pressure is a small fraction of atmospheric pressure, thus any air inside vessel 4 will limit the degree it will compress. For this reason it is preferred to make vessel 4 of metal, as it is difficult to find other flexible materials capable of being hermetically sealed. The wall thickness of vessel 4 is quite thin, typically 0.05-0.1 mm. To increase flexibility, folds and bellows-like shapes can be incorporated. Other geometries can also be used for vessel 4, for example doughnut shape or double-walled pipe shape. Additional spring action can be provided by an internal spring 5 as shown in FIG. 4.

A key to the design is the use of a low “k” highly compressed spring. The “k” is the well-known ratio between the force and the displacement in a spring. Since internal spring 5 has to provide a significant force (resist atmospheric pressure on an area of 20-50 square centimeters, resulting in forces of about 200-500 Newton) while fully compress when the pressure increases by about 100 mm Hg which is about a 13% pressure increase (one atmosphere is 760 mmHg). Assume the spring (which includes the spring constant of vessel 4) has to compress 1 cm for a 100 mmHg increase in blood pressure and the atmospheric pressure on vessel 4 is 400Newton.

The spring constant is: 13%×400 Newton/0.01 meter=5200 Newton/meter. The free length of the spring is 400 Newton/5200 Newton/meter=about 7.6 cm plus the fully compressed length. The fully compressed length can be made as low as 1 mm by using spiral springs that compress to a flat spiral. The vessel 4 provides some of the spring force, however it is desirable to make this a small part of the “k” of the combined spring for two reasons:
1. The walls of vessel 4 should be very thin in order not to use up more than 30% of the elastic limit, and preferable not more than 10%, for a long fatigue life. This provided the vessel with a very low spring constant compared to the internal spring.
2. The preferred embodiment calls for a highly compressed low k spring, which is easier to achieve by an internal spring.

It should be clear that the invention can be practiced without an internal spring simply by using the elasticity of the vessel 4, or by any other means providing volumetric elasticity such as special foams, vessels filled with liquid/gas mixtures or any other device displaying volumetric elasticity. If vessel 4 does not use an internal spring the bellows design shown in FIG. 5 and FIG. 6 should be used, as the free length (without vacuum) or free width of the device should be about 7 times larger than the size after the device was evacuated.

In both FIG. 1 and FIG. 3 the change of volume as a function of the pressure increased can be made linear or not linear, depending on the design of the spring. This behavior can be closely matched to the volume/pressure curve of a healthy aorta or customized for different heart conditions. A simple way to create a non-linear spring is to place two springs in series, each having a different “k”. If the first spring has a spring constant of k1 and the second spring has a constant of k2 (k1>k2), the combined spring has a spring constant of k=1/(1/k1+1/k2) until the second spring is fully compressed, then k=k1.

A customization for a heart condition of particular interest is the use of non-linear volume change to decrease afterload and increase diastolic coronary perfusion in a compromised heart—not unlike an intra-aortic balloon pump. If in FIG. 1 and FIG. 3, at given pressure points, the change in aortic blood volume capacity was not slow but abrupt this could decrease cardiac afterload and increase coronary perfusion independently of blood pressure control. For example, if repeatedly at 100 mmHg blood pressure during cardiac systole aortic blood volume capacity is suddenly increased (FIG. 1 opens widely and FIG. 3 collapses) the resultant sudden decrease in aortic pressure would help the heart to better empty itself into a low pressure system. The result would be an increased stroke volume and minute cardiac output. If repeatedly at a blood pressure of 80 mmHg, during cardiac diastole, there was a sudden decrease in aortic blood volume capacity (FIG. 1 closes and FIG. 3 expands) diastolic blood pressure would increase and augment coronary and renal perfusion.

The internal device also requires means of locating it in the aorta. By the way of example, flexible wires 6 can be compressed when device is inserted via the arteries, then they expand to anchor device in place. Wires 6 can be coated or treated to promote adhesion to aorta walls or the device can be housed within a deployable endovascular stent.

Two alternate embodiments based on a bellows-like structure are shown in FIG. 5 (horizontal bellows) and 6 (vertical bellows). Both have an internal spring 5 and anchoring means 6. The device in FIG. 5 uses a coil (cylindrical) or spiral (taper) spring. FIG. 6 shows a folded flat spring. In order to facilitate the evacuation and sealing of the devices, a pinch-off tube 7 is provided for connection to vacuum pump. The advantage of the bellows configuration over FIG. 3 is that the material is flexed at a lower percentage of the elastic limit. The advantage of FIG. 3 configuration is smoother lines and less chances for deposits forming on the device. The ends of the devices are rounded to avoid damage to blood cells.

The bellows needed for FIG. 5 and 6 are commercially available from suppliers such as Servometer (www.servometer.com) and Alloy Bellows (www.alloybellows.com). The springs are available from most spring suppliers.

What is claimed is:

1. A method for controlling blood pressure comprising adding volumetric elasticity to the blood circulation system without the use of active devices.

2. A method for controlling blood pressure comprising implanting a passive elastic member on the outside of the aorta, said member adding volumetric elasticity to the aorta by making the cross section of the aorta change with blood pressure.

3. A method for controlling blood pressure comprising inserting a passive elastically compressible vessel into the aorta, said vessel elastically decreasing its volume as blood pressure increases.

4. A device for controlling blood pressure by allowing the aorta to elastically increase its volume as blood pressure increase, said device reducing the volume of the blood in the aorta at low blood pressure by deforming the cross section of the aorta from circular to an elongated oval.

5. A passive device for controlling blood pressure inserted into the aorta and capable of elastically increasing the volume of the blood in the aorta by elastically decreasing the volume of said device in response to increasing blood pressure.

6. A devices as in claim 5 wherein said device comprises of an elastic evacuated metal vessel.

7. A device as in claim 5 wherein said device comprises of an elastic evacuated metal vessel containing a spring.

8. A device as in claim 5 wherein the relationship between blood pressure and volume decrease is non-linear.

9. A device as in claim 5 wherein said device is constructed of a sealed and evacuated metal bellows.

10. A method of assisting the pumping function of the heart, method comprising of adding volumetric elasticity to the blood circulation system without the use of active devices.

11. A method as in claim 10 wherein said volumetric elasticity is achieved by a spring compressing the aorta.

12. A method as in claim 10 wherein said volumetric elasticity is achieved by a non-linear spring compressing the aorta.

13. A method as in claim 10 wherein said volumetric elasticity is achieved by the insertion of a compressible object into the aorta.

14. A method as in claim 10 wherein said volumetric elasticity is achieved by the insertion of a compressible object into the aorta, volume of said object changing with pressure in a non-linear manner.
